(19) United States
(12) Patent Application Publication
(10) Pub. No.: US 2009/0299244 A1
(43) Pub. Date: Dec. 3, 2009

(54) KNEE BRACE FOR REHABILITATION

(75) Inventors: Pang-Ching Chiang, Tainan City (TW); Chien-Huei Lee, Tainan City (TW)

Correspondence Address: Stevens Law Group, 1754 Technology Drive, Suite #226, San Jose, CA, 95110, US

(73) Assignee: Plus Meditech Co., Ltd., Tainan City (TW)

(21) Appl. No.: 12/127,632

(22) Filed: May 27, 2008

(51) Int. Cl. A61F 5/00

(52) U.S. Cl. 602/26; 602/16

(57) ABSTRACT

A knee brace for rehabilitation provides a stable support for an injured knee using a four-crank mechanism. Positioning pins are used to fix limiting blocks pivoted to the upper and lower braces. It limits the moving range of the crank between the upper and lower braces, restricting the moving angles of the upper and lower braces. Besides, the buffer pads are covered at the pivotal axes of the upper and lower braces in order to alleviate the collision between joint surfaces during the motion of injured knee. Such a mechanism protects the rehabilitating knee joint. The disclosed structure can solve the failure problem of the knee brace for rehabilitation due to a loose pin in the prior art. The counterforce to injured knee from the performance of knee brace reduced for rehabilitation can prevent the user's knee joint from further injuries.

### BACKGROUND OF THE INVENTION

1. Field of Invention

The invention relates to a joint brace for rehabilitation and, in particular, to a knee brace for rehabilitation that uses positioning pins to fix limiting blocks, thereby controlling the motion ranges of upper and lower braces connected by a crank.

2. Related Art

Joints connect bones in different portions of the human body. Unlike the middle sections of other bones, the joints do not have the protection of muscles and fats. Therefore, when the joint is hit with an external force, it can be seriously damaged. In particular, the knee joints carry the most burdens. It has a much larger probability to be damaged or age than other joints.

When the knee joint is just hurt, the patient has to reduce or avoid activities. Only after the bone recovers can the patient gradually increase the moving angle of the knee joint. Therefore, rehabilitation tools are often required as an aide. Such a knee brace usually involves connecting mechanisms such as a crank mechanism, a cam mechanism, and a gear mechanism. Take a four-crank knee brace as an example. It usually comprises an upper brace fixed to the thigh, a lower brace fixed to the leg, and a crank connected between the upper and lower braces. An angle-adjusting mechanism is provided between the upper and lower braces. Usually, a pin is directly inserted on the motion track of the crank to restrict its motion range, thereby limiting the moving angle between the upper and lower braces.

However, the pin is too small for holding. It is thus very inconvenient to adjust the motion angle between the upper and lower braces. Moreover, the pin is likely to get lost. Currently, the pin is simply inserted on the motion track of the crank, and there is no mechanism for fixing the pin. Once the pin is dropped from the angle-adjusting mechanism, the knee brace loses the function of restricting the motion angle of the knee joint. As a consequence, the user's knee joint may be hurt again because the moving angle of the knee is too large.

### SUMMARY OF THE INVENTION

In view of the problem that the four-crank knee brace is likely to lose the function of restricting the moving angle of the knee joint because the pin gets lost, the invention discloses a knee brace for rehabilitation. The invention can restrict the moving angle of knee joint, protecting the user's knee joint from further injuries because of the restriction function failure.

The invention provides a knee brace for rehabilitation. It includes a fixing base, an upper brace, a lower brace, a first crank, a first limiting block, a second limiting block, a front sheltering plate, and two positioning pins. One end of the upper brace has a first pivotal axis and a second pivotal axis. The upper brace is pivoted to the fixing base through the first pivotal axis. One end of the lower brace has a third pivotal axis and a fourth pivotal axis. The lower brace is pivoted to the fixing base through the third pivotal axis. One end of the first crank is pivoted at the second pivotal axis of the upper brace. The other end is pivoted at the fourth pivotal axis of the lower brace.

The first limiting block has several first positioning holes, and is pivoted at the first pivotal axis of the upper brace. It is used to restrict the motion range of the end of the first crank that is pivoted to the upper brace. The second limiting block has several second positioning holes. The second limiting block is pivoted at the third pivotal axis of the lower brace. It is used to restrict the motion range of the end of the first crank that is pivoted to the lower brace. The front sheltering plate has several first through holes and several second through holes. The front sheltering plate covers the first crank, the first limiting block, and the second limiting block. The two positioning pins go through the first through hole on the front sheltering plate and the corresponding first positioning hole and the second through hole on the front sheltering plate and the corresponding second positioning hole, respectively. This fixes the relative positions of the first limiting block and the second limiting block with respect to the fixing base.

One difference between the disclosed knee brace for rehabilitation and the prior art is that the invention uses a positioning pin to fix the relative positions between the limiting blocks on the upper and lower braces and the fixing base. By controlling the motion range of the crank between the upper and lower braces, the moving angles of the upper and lower braces are limited to a certain range. Using such measures, the invention can solve the failure problem of the knee brace for rehabilitation due to a loose pin in the prior art. The reduction of the counterforce forced to the knee joint from the knee brace for rehabilitation can prevent the user's knee joint from further injuries.

### BRIEF DESCRIPTION OF THE DRAWINGS

The invention will become more fully understood from the detailed description given herein below illustration only, and thus is not limitative of the present invention, and wherein:

FIG. 1A is an exploded view of the preferred embodiment of the disclosed knee brace for rehabilitation;

FIG. 1B is a schematic view showing the assembled knee brace for rehabilitation according to the invention;

FIG. 2 is a schematic view of the upper and lower braces in the disclosed knee brace for rehabilitation;

FIG. 3 is a schematic view of a first embodiment of the disclosed knee brace for rehabilitation where the limiting value of extension angle is set to be 150 degrees; and

FIG. 4 is a schematic view of a second embodiment of the disclosed knee brace for rehabilitation where the limiting value of extension angle is set to be 120 degrees.

### DETAILED DESCRIPTION OF THE INVENTION

The present invention will be apparent from the following detailed description, which proceeds with reference to the accompanying drawings, wherein the same references relate to the same elements.

The disclosed knee brace for rehabilitation utilizes limiting blocks to restrict the motion range of the crank between upper and lower braces, thereby restricting the moving angles of the upper and lower braces. The following embodiments along with the companying figures explain how the invention is implemented, but should not be used to limit the scope of the invention defined in the claims hereinafter.

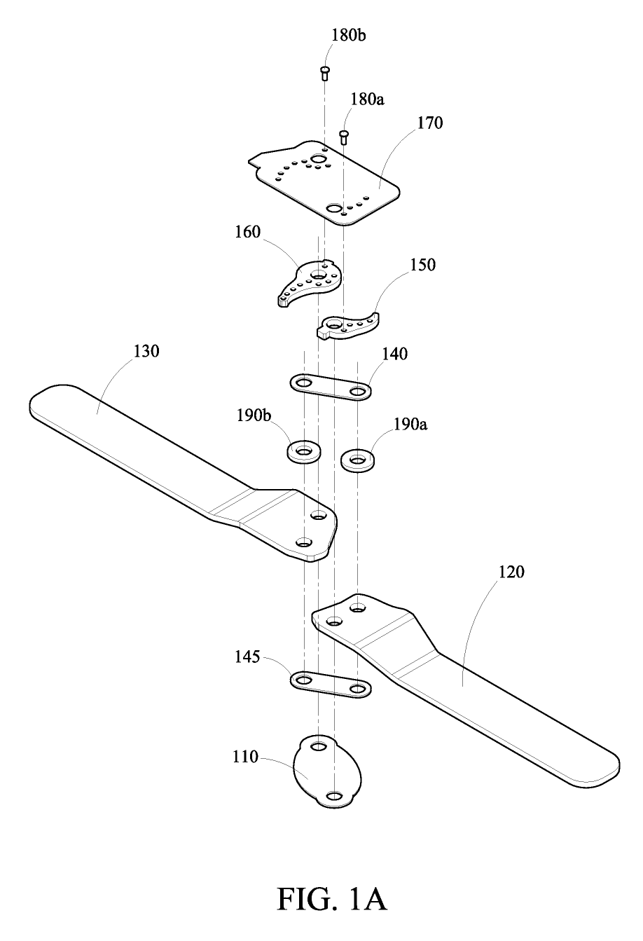

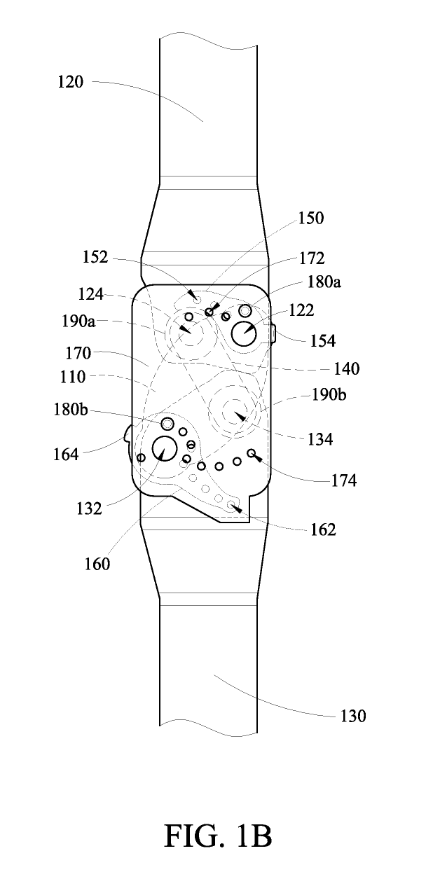

FIG. 1A is an exploded view of the disclosed knee brace for rehabilitation. FIG. 1B is a schematic view showing the assembled knee brace for rehabilitation according to the invention. Please refer to FIGS. 1A and 1B simultaneously. The knee brace for rehabilitation 100 includes a fixing base 110, an upper brace 120, a lower brace 130, a first crank 140, a first limiting block 150, a second limiting block 160, a front sheltering plate 170, a positioning pin 180a and another positioning pin 180b. The upper brace 120 is fixed to the thigh of the user to move with the motion thereof. One end of the upper brace 120 has a first pivotal axis 122 and a second pivotal axis 124. The upper brace 120 is pivoted to the fixing base 110 through the first pivotal axis 122. The lower brace 130 is fixed to the leg of the user to move with the motion thereof. One end of the lower brace 130 has a third pivotal axis 132 and a fourth pivotal axis 134. The lower brace 130 is pivoted to the fixing base 110 through the third pivotal axis 132.

On the other hand, the ends of the first crank 140 are pivoted at the second pivotal axis 124 of the upper brace 120 and the fourth pivotal axis 134 of the lower brace 130 respectively. Therefore, the fixing base 110, the upper brace 120, the lower brace 130, and the first crank 140 form a four-crank mechanism. When the upper brace 120 rotates with respect to the first pivotal axis 122, the lower brace 130 is driven by the first crank 140 to rotate with respect to the third pivotal axis 132.

In this embodiment, the sizes of the fixing base 110, the upper brace 120, the lower brace 130, and the first crank 140 and the positions of the pivots 122, 124, 132, 134 are designed according to the rotation ratio between thigh and leg in human's biomechanics. Thus, the invention can simulate the actual rotation mechanism of the knee joint.

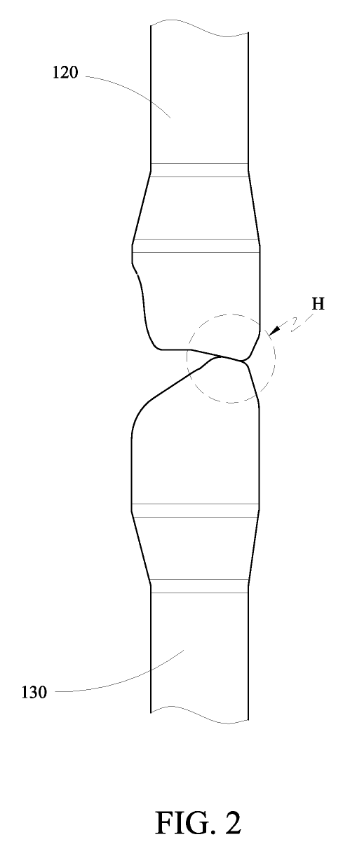

It is worth mentioning that when the angle between the upper brace 120 and the lower brace 130 is 180 degrees, one end of the upper brace 120 against one end of the lower brace 130, as indicated by point H in FIG. 2. In other words, the disclosed knee brace for rehabilitation 100 restricts the maximal angle between the upper brace 120 and the lower brace 130 to 180 degrees using the end profile feature thereof. This prevents the user from hyper-extension at the knee joint.

Besides, the knee brace for rehabilitation 100 may further include a second crank 145. One end of the second crank 145 is pivoted at the second pivotal axis 124 of the upper brace 120 between the upper brace 120 and the fixing base 110. Its other end is pivoted at the fourth pivotal axis 134 of the lower brace 130 between the lower brace 130 and the fixing base 110. It should be noted that the second crank 145 is mainly used to increase the entire structure rigidity. Therefore, a person skilled in the art can determine whether to include the second crank 145 according to the actual situation. For example, the combination of the upper brace 120, the lower brace 130, and the first crank 140 already has certain rigidity. In most cases, one can opt not to include the second crank 145 in the disclosed knee brace for rehabilitation 100.

Please further refer to FIGS. 1A and 1B. The first limiting block 150 has several first positioning holes 152. It is pivoted at the first pivotal axis 122 of the upper brace 120. That is, the first limiting block 150 can rotate with respect to the first pivotal axis 122. The second limiting block 160 is pivoted at the third pivotal axis 132 of the lower brace 130, and can rotate with respect to the third pivotal axis 132. The second limiting block 160 has several second positioning holes 162.

The front sheltering plate 170 has several first through holes 172 and several second through holes 174. It covers the first crank 140, the first limiting block 150, and the second limiting block 160. Besides, the first limiting block 150 can rotate with respect to the first pivotal axis 122, so that one of the first positioning holes 152 on the first limiting block 150 corresponds to one of the first through holes 172 on the front sheltering plate 170. In this embodiment, the first limiting block 150 has a first adjusting part 154, for example. The first adjusting part 154 is exposed outside the front sheltering plate 170, so that the user can rotate the first limiting block 150 using the first adjusting part 154.

On the other hand, the second limiting block 160 can rotate with respect to the third pivotal axis 132, so that one of the second positioning holes 162 on the second limiting block 160 corresponds to one of the second through holes 174 on the front sheltering plate 170. The second limiting block 160 in this embodiment also has a second adjusting part 164 exposed out of the front sheltering plate 170, so that the user can rotate the second limiting block 160 using the second adjusting part 164.

The positioning pin 180a is inserted into one of the first through holes 172 of the front sheltering plate 170 and the corresponding first positioning hole 152, thereby fixing the relative position of the first limiting block 150 with respect to the fixing base 110. As a result, the first limiting block 150 can restrict the motion range of the end of the first crank 140 connecting to the upper brace 120. The positioning pin 180b is inserted into one of the second through holes 174 of the front sheltering plate 170 and the corresponding second positioning hole 162, thereby fixing the relative position of the second limiting block 160 with respect to the fixing base 110. In this case, the motion range of the end of the first crank 140 connecting to the lower brace 130 is restricted by the second limiting block 160.

In this embodiment, the positioning pins 180a, 180b can be screws. In this case, the first positioning hole 152 of the first limiting block 150 has a screw thread for the positioning pin 180a to go through the first through hole 172 of the front sheltering plate 170 and get locked in the first positioning hole 152. The second positioning hole 162 of the second limiting block 160 can also have a screw thread for the positioning pin 180b to go through the second through hole 174 of the front sheltering plate 170 and get locked in the second positioning hole 162.

In practice, by inserting the positioning pin 180a into different first positioning holes 152 and the corresponding first through holes 172 on the front sheltering plate 170, the swinging angle (the angle between the upper brace 120 and the lower brace 130, usually called the extension angle) of the upper brace 120 can have different limiting values. In the embodiments of the invention, the limiting angles can be 150, 160, 170, or 180 degrees.

Likewise, by inserting the positioning pin 180b into different second positioning holes 162 and the corresponding second through holes 174 on the front sheltering plate 170, the swinging angle of the lower brace 130 (the angle between the lower brace 130 and the upper brace 120, usually called the flexion angle) can have different limiting values. IN the embodiments of the invention, the limiting angles can be 0, 10, 20, 30, 45, 60, 75, 90, 105, or 120 degrees.

To avoid the reaction due to the collisions between the first crank 140 and the first limiting block 150 and the second limiting block 160 from hurting the knee joint of the user, a first buffer pad 190a and a second buffer pad 190b are covered at the second pivotal axis 124 of the upper brace 120 and the fourth pivotal axis 134 of the lower brace 130, respectively. Their function is to absorb the reaction due to the collisions between the first crank 140 and the first limiting block 150 and the second limiting block 160.

From the above-mentioned embodiment, it is clear that the disclosed knee brace for rehabilitation utilizes positioning pins to fix the relative positions of the first limiting block and the second limiting block with respect to the fixing base. This controls the motion range of the first crank, thereby restricting the moving angles of the upper and lower braces. Moreover, the paramedics can adjust the positions of the first and second limiting blocks according to the actual situation of the patient's knee joint. In this case, the moving angles of the upper and lower braces have different limiting values due to the different positions of the first and second limiting blocks. Using the invention, the user's knee joint is allowed to move within an appropriate range to avoid hyper-extension and further injuries before the knee joint is completely recovered.

Besides, the material of the disclosed knee brace can be metal or plastic. So long as the required structural strength is met, the invention can use any possible material. It is not limited by the above-mentioned examples.

For those skilled in the art to better understand the invention, the action of the disclosed knee brace for rehabilitation 100 is further elucidated with reference to the accompanying figures.

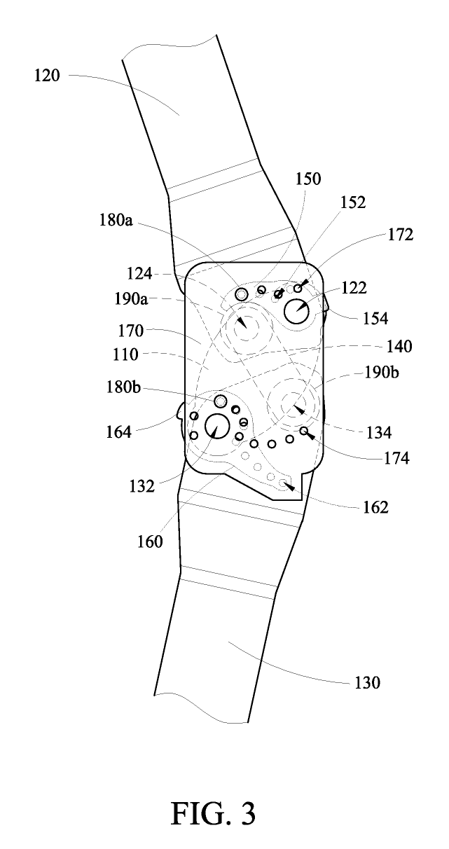

FIG. 3 is a schematic view of a first embodiment of the disclosed knee brace for rehabilitation where the limiting value of extension angle is set to be 150 degrees. As shown in the drawing, if the paramedic evaluates the injured knee joint (anterior cruciate ligament, so-called “ACL”, injury, posterior cruciate ligament, so-called “PCL” injury, or osteoarthritis, so-called “OA”) of the patient and determines that the joint can extend by 150 degrees at most, he or she can use the first adjusting part 150 of the first limiting block 150 to rotate the first limiting block 150 with respect to the first pivotal axis 122 so that the first positioning hole 152 corresponding to the 150-degree limiting extension angle corresponds to the first through hole 172 on the front sheltering plate 170. The positioning pin 180a then goes through the first through hole 172 and gets fixed in the first positioning hole 152. In this case, the first limiting block 150 and the fixing base 110 are fixed as shown in the drawing. As the upper brace 120 swings with the user's thigh, it can subtend at most 150 degrees with respect to the lower brace 130. That is, the user can only extend his or her knee joint to the extent that the angle between the upper brace 120 and the lower brace 130 is 150 degrees. Therefore, the disclosed knee brace for rehabilitation 100 can indeed prevent the user's knee joint from extending beyond the limit.

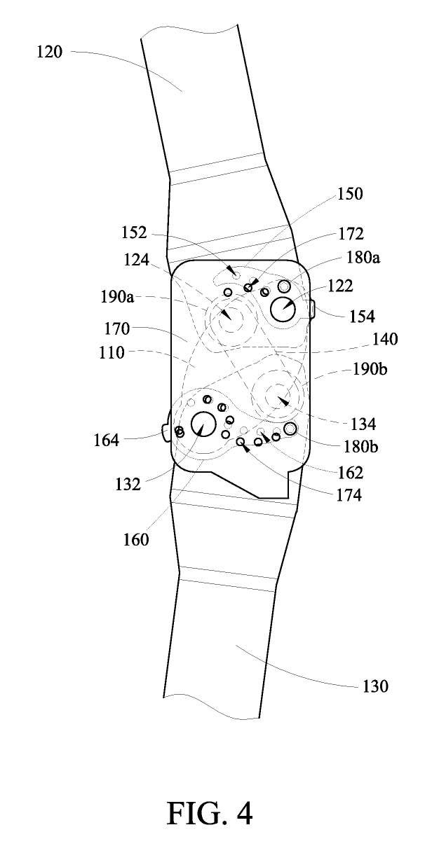

FIG. 4 is a schematic view of a second embodiment of the disclosed knee brace for rehabilitation where the limiting value of extension angle is set to be 120 degrees. As shown in the drawing, if the paramedic evaluates the injured knee joint of the patient and determines that the joint can extend by 120 degrees at most, he or she can use the first adjusting part 154 of the second limiting block 160 to rotate the second limiting block 160 with respect to the third pivotal axis 132 so that the second positioning hole 162 corresponding to the 120-degree limiting flexion angle corresponds to the second through hole 174 on the front sheltering plate 170. The positioning pin 180b then goes through the second through hole 174 and gets fixed in the second positioning hole 162. In this case, the second limiting block 160 and the fixing base 110 are fixed as shown in the drawing. As the lower brace 130 swings with the user's leg, the angle between it and the upper brace 120 is at least 120 degrees. That is, the user can only bend his or her knee joint to the extent that the angle between the upper brace 120 and the lower brace 130 is 120 degrees. Therefore, the disclosed knee brace for rehabilitation 100 can indeed prevent the user's knee joint from extending beyond the limit.

In summary, the invention utilizes a four-crank mechanism to provide a stable support for an injured knee. Positioning pins are used to fix the relative positions of the limiting blocks pivoted to the upper and lower braces with respect to the fixing base. This structure controls the motion ranges of the crank pivoted between the upper and lower braces, thereby restricting the moving angles of the upper and lower braces. Besides, the buffer pads are covered at the pivotal axes of the upper and lower braces in order to alleviate the collision between joint surfaces during the motion of injured knee. Such a mechanism protects the rehabilitating knee joint. The disclosed structure can solve the failure problem of the knee brace for rehabilitation due to a loose pin in the prior art. The reduction of the counterforce forced to the knee joint from the performance of knee brace for rehabilitation can prevent the user's knee joint from further injuries.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. A knee brace for rehabilitation, comprising:
a fixing base;
an upper brace, which has a first pivotal axis and a second pivotal axis on its one end and is pivoted to the fixing base the through first pivotal axis;
a lower brace, which has a third pivotal axis and a fourth pivotal axis on its one end and is pivoted to the fixing base through the third pivotal axis;
a first crank, one end of the first crank is pivoted at the second pivotal axis of the upper brace and the other end is pivoted at the fourth pivotal axis of the lower brace;
a first limiting block, which has a plurality of positioning holes and is pivoted at the first pivotal axis of the upper brace for limiting the motion range of the end of the first crank pivoting to the upper brace;
a second limiting block, which has a plurality of positioning holes and is pivoted at the third pivotal axis of the lower brace for limiting the motion range of the end of the first crank pivoting to the lower brace;
a front sheltering plate, which has a plurality of first through holes and a plurality of second through holes and covers the first crank, the first limiting block and the second limiting block; and
two positioning pins, which are respectively inserted into one of the first through holes on the front sheltering plate and the corresponding first positioning hole and one of the second through holes and the corresponding second positioning hole to fix the positions of the first limiting block and the second limiting block relative to the fixing base.

2. The knee brace for rehabilitation of claim 1 further comprising a second crank, one end of the second crank is pivoted at the second pivotal axis of the upper brace between the upper brace and the fixing base, and the other end of the second crank is pivoted at the fourth pivotal axis of the lower brace between the lower brace and the fixing base.

3. The knee brace for rehabilitation of claim 1 further comprising:
a first buffer pad, which is mounted at the second pivotal axis of the upper brace and between the first crank and the upper brace; and
a second buffer pad, which is mounted at the fourth pivotal axis of the upper brace and between the first crank and the lower brace.

4. The knee brace for rehabilitation of claim 1, wherein an end edge of the upper brace against an end edge of the lower brace when the angle between the upper brace and the lower brace is 180 degrees.

5. The knee brace for rehabilitation of claim 1, wherein the first limiting block further has a first adjusting part exposed out of the front sheltering plate.

6. The knee brace for rehabilitation of claim 5, wherein the second limiting block further has a second adjusting part exposed out of the front sheltering plate.

7. The knee brace for rehabilitation of claim 1, wherein when one of the positioning pins is inserted into one of the first positioning holes the angle between the upper brace and the lower brace has a maximum of 150, 160, 170, or 180 degrees.

8. The knee brace for rehabilitation of claim 1, wherein when one of the positioning pins is inserted in one of the second positioning holes the angle between the upper brace and the lower brace has a minimum of 0, 10, 20, 30, 45, 60, 75, 90, 105, or 120 degrees.
